(19) United States
(12) Patent Application Publication
(10) Pub. No.: US 2004/0199404 A1
(43) Pub. Date: Oct. 7, 2004

(54) Integrated system and method for documenting and billing patient medical treatment and medical office management

(76) Inventors: Bart Ripperger, Phoenix, AZ (US); Joseph H. Miglietta, Scottsdale, AZ (US); William Reese, Tempe, AZ (US)

Correspondence Address: WEISS & MOY PC, 4204 NORTH BROWN AVENUE, SCOTTSDALE, AZ, 85251, US

(21) Appl. No.: 10/406,076

(22) Filed: Apr. 2, 2003

(51) Int. Cl. G06F 17/60; A61B 5/00

(52) U.S. Cl. 705/2; 600/300

(57) ABSTRACT

A method in a computer system for organizing a medical facility is disclosed. The method comprises: scheduling a visit by a patient; entering and storing medical history of the patient in a database; retrieving the medical history of the patient from the database during the visit by the patient; diagnosing patient based on information entered into the computer system; and billing the patient.

### BACKGROUND OF THE INVENTION

1. Field of the Invention

This invention relates generally to medical office management and, more specifically, to an integrated system and method for documenting and billing patient medical treatment and medical office management.

2. Description of the Prior Art

As healthcare cost increase throughout the world, healthcare providers must make difficult choices regarding how to provide the best possible services at prices their patients can afford. Although most clinicians would prefer to purchase every cutting edge medical technology available, they simply cannot afford to do so while still caring for their patients in an affordable manner.

The main problem is that there are too many areas in which the physician is responsible for in order for a medical office to run properly. A physician is responsible for starting a medical record/medical history for a patient, taking chart notes/creating medical records, billing, maintaining inventory/office supplies, prescriptions, outcome studies, etc. While a physician may have others help in maintaining these different areas, ultimately, the physician needs to be aware of and is responsible for maintaining them.

To maintain all of the above is a very time consuming process. Many of the above areas need to be taken care of manually. While there are some software programs to help individually manage the above areas, none of them are compatible so that one software program will manage an entire medical office. Listed below are ways of maintaining the above mentioned areas.

When creating patient history (personal information and medical/surgical history), patients generally fill out personal and medical history information forms. This information is then entered manually into the patient file and record by office staff into the office computer system. This is a slow, time consuming, labor intensive process for the office staff/employees. There is presently no software available to record and store this information.

Since the onset of modern medical treatment, there has been a need to maintain accurate records of patient treatment. The information maintained in a patient treatment record is what the physician relies on to carry out a course of treatment, and also to justify the billing for service. The increasing demands placed on a physician's time results too often in patient charts that are improperly maintained or at best difficult to read. The requirement for accurate patient records, while mandatory by law, is necessary for good patient treatment but also for determining the efficacy of patient treatment. It is the latter reason that is becoming increasingly in the spotlight as expensive treatments are being scrutinized for their effectiveness.

The systems and procedures currently in use for creating chart notes and medical records are handled in several ways including: 1) the physician handwriting individual chart notes; or 2) the physician dictating into a recording device the details of the encounter, then this information being transcribed into a note for the medical record. These methods have proven to be very slow, time consuming and labor intensive.

More recent developments and alternatives have included computer programs that attempt to produce medical records and chart notes for physicians. These include: 1) voice dictation; 2) computer constructed chart notes base on information a physician can construct to his own personal specifications for content, but that must work through a pre-designed flow pattern to build a chart note/medical record. These applications use a spread sheet that corresponds to a practice superbill to allow the physician to check off boxes that correspond to the treatment administered. Edit fields are available where the physician can type in information related to the specifics of the symptoms observed or to the treatment administered. While useful for gathering and storing information into a database for future retrieval, such systems are cumbersome to use and require a physician to be tied to a computer. Because this is not possible during the patient exam or treatment, the physician is still left to rely on hand written notes that are subsequently entered into the spread sheet. This has the obvious flaws of causing the possibility of inaccurate information and loss of efficiency of the physician's time. These systems also require the physician to continue frequently repetitive tasks and repeating the same information numerous times to complete a chart note. These systems further require the physician to repetitively fill in specific fields to build a complete chart note. They have also proven to be very cumbersome and lack flexibility to allow the physician to work in a seamless, unrestricted manner. Furthermore, these systems also require the physician to enter and build the content/intellectual property to support each set of clinical findings and diagnoses as well as treatments (procedures, medications, surgeries, supplies etc.) he utilizes.

In order to bill a client, a physician has traditionally completed a superbill/patient encounter form after a patient's visit. This superbill has the diagnosis (ICD) code and the procedure, surgery or E&M code (CPT code) which describe the details of the encounter required for billing the patient or insurance company. The office staff, then fills out the insurance claim form (the HCFA 1500 form) manually for billing the insurance company, or the information and codes are entered manually by the office staff into a computer software system which then creates a patient file. The office staff then can enter the appropriate billing codes into the insurance claim form (HCFA 1500) which is part of the computer system. This can then either be printed out and mailed to the insurance company or sent electronically to the insurance company. All of this process is very time consuming and labor intensive for office staff and expensive to the physician to pay for the man hours and labor to perform these tasks required for billing. Further, none of the software for billing is compatible with the software for the starting and maintaining of medical records. Thus, one cannot easily transport information from the software for the starting and maintaining of medical records to any existing billing software.

When a physician deems necessary, various supplies are dispensed to a patient. Some of the supplies that may be dispensed on any given office encounter include, but is not limited to, splints, casts, fracture orthoses, pads bandages and dressings, orthotic devices, braces, etc. Currently these items are dispensed/given to a patient with virtually no communication to office staff/billing employees other than recording these on a superbill, which frequently can erroneously miss the supply resulting in missed billing for the supply and failure by the office to reorder and restock the utilized supplies.

Frequently patients will require prescription medication from a physician for appropriate treatment of a medical problem. Currently these are hand written on a paper prescription form by the doctor and given to the patient to take to a pharmacy to be filled and dispensed. This method is slow. It is very labor intensive to hand write every prescription. Also errors can occur at the pharmacy due to inability of the pharmacist to read the handwriting of the physician resulting in medication and dosing errors for patients. Additionally, patients frequently lose the paper prescription and consequently never obtain their necessary medication.

With the ever increasing cost of health care, surgeries, medications etc., it has become necessary to find means to justify cost and efficacy of medical treatments. Until now very little can be done to identify and justify costs and efficacy of treatments. Random studies can be done in teaching hospital settings for studies on procedures. Attempts have been made to retrieve data from multiple physician offices to try to study effectiveness of various treatments and procedures.

Therefore, a need existed to provide an integrated system and method for documenting and billing patient medical treatment. The system would be an easy-to-use, accurate, secure system to facilitate the recording of patient treatment information. The system should be minimally intrusive to a physician's manner of work thereby allowing the physician to maximize time spent with patients. The system should be a fully integrated system which will allow a physician to perform the following tasks: starting a medical record/medical history for a patient file; taking chart notes/creating medical records; billing; maintaining inventory/office supplies; prescriptions; outcome studies; and combinations thereof.

### SUMMARY OF THE INVENTION

In accordance with one embodiment of the present invention, it is an object of the present invention to provide an integrated system and method for documenting and billing patient medical treatment.

It is another object of the present invention to provide a system that would be an easy-to-use, accurate, secure system to facilitate the recording of patient treatment information.

It is still another object of the present invention to provide a system that will be minimally intrusive to a physician's manner of work thereby allowing the physician to maximize time spent with patients.

It is another object of the present invention to provide a system that will be a fully integrated system which will allow a physician to perform the following tasks: starting a medical record/medical history for a patient file; taking chart notes/creating medical records; billing; maintaining inventory/office supplies; prescriptions; outcome studies; and combinations thereof.

### BRIEF DESCRIPTION OF THE EMBODIMENTS

In accordance with one embodiment of the present invention, a method in a computer system for organizing a medical facility is disclosed. The method comprises: scheduling a visit by a patient; entering and storing medical history of the patient in a database; retrieving the medical history of the patient from the database during the visit by the patient; entering the diagnosis for the patient based on knowledge and observations of physician; entering any supporting facts and observations; entering treatment administered to the patient during the visit; entering facts related to the treatment; entering the treatment plan; reviewing associated diagnosis/treatment risks with the patient and recording the patient consent for treatment; and billing the patient.

In accordance with another embodiment of the present invention, a computer readable medium containing instructions for controlling a computer system for organizing a medical facility is disclosed. The computer readable medium controls the computer system by: scheduling a visit by a patient; entering and storing medical history of the patient in a database; retrieving medical history of the patient from the database during the visit by the patient by downloading the medical history of the patient from the computer system to a portable electronic device; entering the diagnosis for the patient based on knowledge and observations of physician; entering any supporting facts and observations; entering treatment administered to the patient during the visit; entering facts related to the treatment; entering the treatment plan; reviewing associated diagnosis/treatment risks with the patient and recording the patient consent for treatment; billing the patient; maintaining an inventory of office supplies in the medical facility; and preparing a prescription form to be given to the patient.

The foregoing and other objects, features, and advantages of the invention will be apparent from the following, more particular, description of the preferred embodiments of the invention, as illustrated in the accompanying drawings.

### BRIEF DESCRIPTION OF THE DRAWINGS

The novel features believed characteristic of the invention are set forth in the appended claims. The invention itself, as well as a preferred mode of use, and advantages thereof, will best be understood by reference to the following detailed description of illustrated embodiments when read in conjunction with the accompanying drawings.

FIG. 1 is a schematic of the patient treatment model.

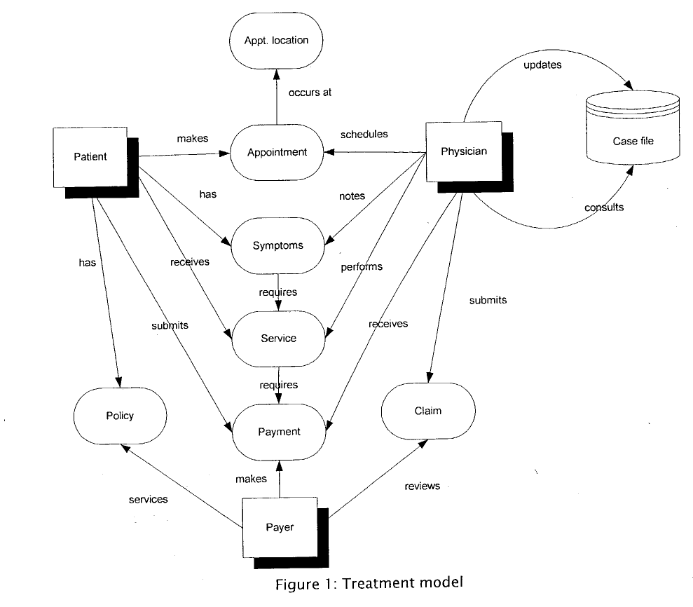

FIG. 2 is a schematic of the flow path used for treatment using the physician assistant.

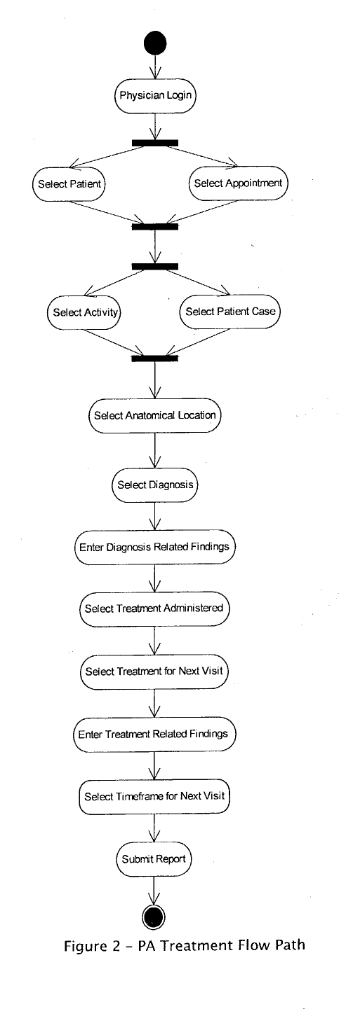

FIG. 3 is a diagram of the diagnosis picker.

FIG. 4 is a diagram of the anatomy picker.

FIG. 5 is a simplified functional block diagram of a system using the software of the present invention.

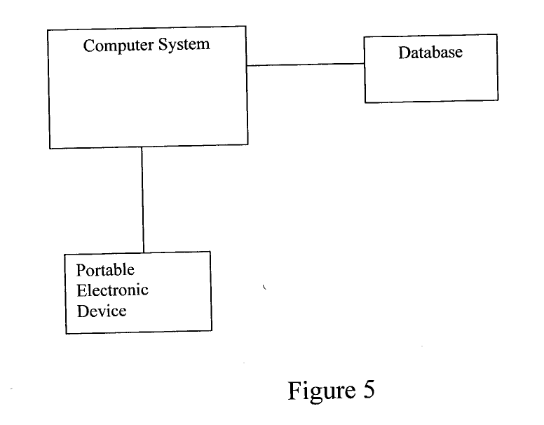

FIG. 6 is a flow chart depicting the operation of the software of the present invention.

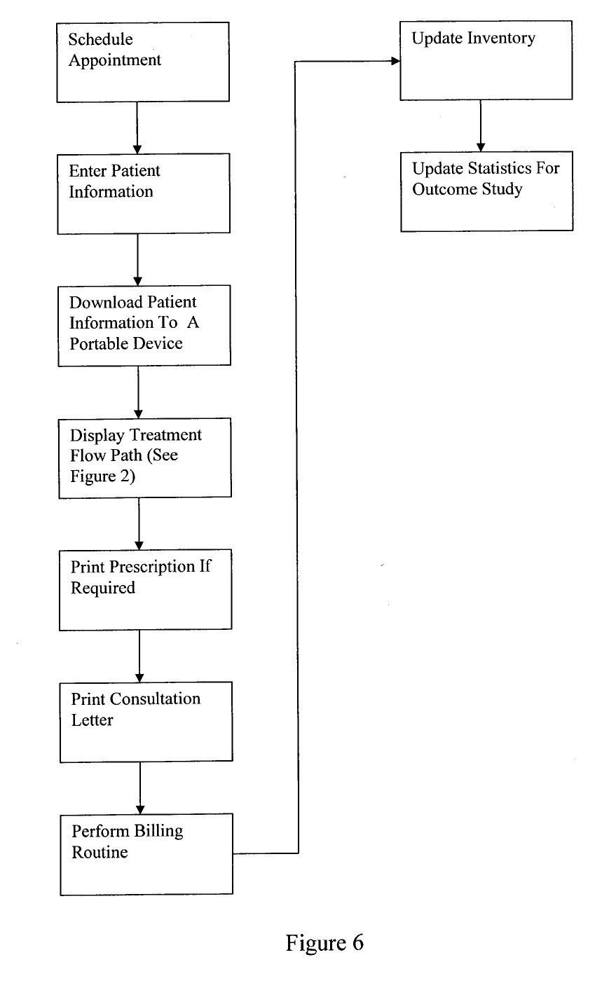

### DETAILED DESCRIPTION OF THE PREFERRED EMBODIMENT

The present invention provides an integrated system and method for facilitating the documentation and billing of patient medical treatment. The system which is accurate, easy-to-use, and secure, provides a fast and efficient means of documenting each interaction the physician has with the patient.

Referring to the Figures details of the software will be disclosed. The software will have different module components. Each of the modules can interact with other modules and can function independently or in combination with one another. The software will have a front office patient management module. The front office patient management module schedules the patient's visit. Scheduling the patient visit includes but is not limited to, specifying a patient's name, an office visit date and time, a preferred physician and location. The software handles additional tasks associated with a schedule visit. These tasks include, but are not limited too, reserving any resources required for treatment, ordering supplies for treatment, and reserving a physician's time. Resources includes items such as the examination/treatment room, treatment equipment, supplies, and reserving assisting personnel time. If the patient has a chief complaint this information can also be entered. The software is designed to be flexible to allow one to enter information by various means amongst which includes, but is not limited to, keyboards entry through the use of a secure internet web site, handwriting recognition using a tablet computer, pressing the numeric numbers on a touch tone telephone in response to a prompt or by audibly entering the information coupled with automatic speech recognition capability to record any answers. Office personnel may alternatively enter this information manually into the software. In general, the software is designed to ask a plurality of pre-recorded questions. The patient may then enter an answer to the question by one of the above manners. If the date, time, physician, etc. is not available, the software will notify the patient. In general, the software will schedule a time for an appointment to the closest available opening to which the patient requested. This information is then stored in an information database.

If the patient is new to the practice, a patient history is entered. The patient history will include, but is not limited too, such information such as history of prior illnesses, family illnesses, allergies, current medications, etc. The software is designed to allow this information to be entered in several different manners. Various means of data entry are supported that range from direct electronic recording from patient entry to manual key input by office personnel. For example, a prerecorded message may go through a plurality of questions for the new patient to answer. The patient may push numbers on the telephone to answer the questions. Alternatively, the patient may give audible answers to the questions. The software is designed with automatic speech recognition capability to record the answers. Office personnel may further enter this information manually into the software. Once this information is entered, the data is stored in the information database.

On the date of visit, the patient and appointment information is transferred to a physician's assistant (PA). The PA is the preferred tool or means of data entry. The PA can range from an electronic personal data assistant (PDA), tablet with a wireless network link (WLAN), a networked desktop/laptop computer, and the like. It should be noted that the listing of different PA devices should not be seen as to limit the scope of the present invention. Other types of PAs may be used without departing from the spirit or scope of the present invention. From the PA the physician can determine who the patient is by looking up the next patient on the roster, or appointment block, or recognize the patient from a scanned photo that appears on the PA. The entire patient medical history, chief complaint, and treatment history are available for viewing since all of this information is downloaded to the PA. The schedule screen may color code the patient's information which would show types of insurance, nature of office visit, etc. Furthermore, the software may be designed so that when a patient's name or phone number is “clicked”, the software will automatically dial the number.

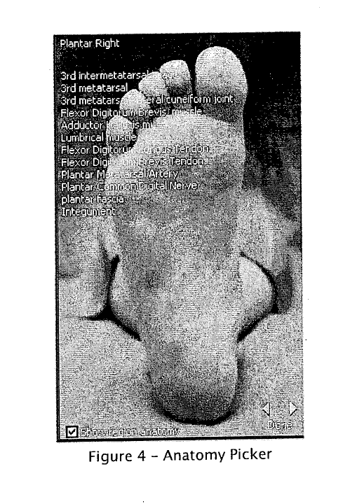

During or after the examination, the physician may enter and store notes on the PA. The software is designed to allow the physician to input information during and after the examination. Information may be entered in a plurality of different manners. For example, pull down menus are provided for allowing the physician to easily input information during and after the examination. The pull down menus will have information which the physician may “click on” to enter information about the patient into the PA. The software will further have visual means for entering examination information (See FIG. 4). For example, after examining the patient the physician documents the area of the pain by selecting the location from an anatomy photo which is displayed on the PA.

In accordance with one embodiment of the present invention, the visual means is an anatomical image that contains complete mapping of every anatomical structure/tissue (integument, nerve, vessel, bone, muscle, tendon and ligament) that is located within a region on the image. The zones/regions are built into the software and are present on the server side of the image only. The user simply sees the anatomical image, and when a mouse/cursor or stylus is passed over any particular area the user simultaneously sees a list of every anatomical structure that is located within a given region. This anatomical list automatically changes as the cursor is moved over various regions to accurately convey all anatomical structures found within the area of the cursor. When the physician determines the area of pain or pathology a patient describes, the physician then selects that anatomical location and tissue type from the anatomy picker. Once this area of pathology is selected by the physician, it is then brought into the medical record and is stored as the pathological area and can be referenced in the note/medical record at a multitude of locations wherever accurate description of pathological area is appropriate and necessary.

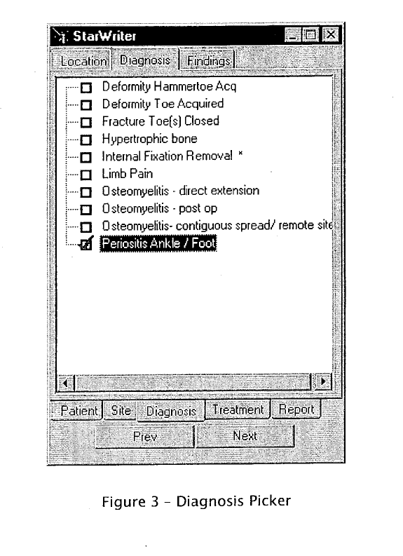

After selecting an area on the anatomy, a menu (or a new screen) will appear which will show different illnesses from which the patient may be suffering from (See FIG. 3). The list is generated from a database table that relates illnesses to tissue type or anatomical location. Each illness may show different symptoms to help the physician with his/her diagnosis. Thus the software will help the physician with the proper diagnosis. Any of the symptoms listed may be added to the patient's file by simply “clicking” the symptom. Thus, the software allows the physician to easily enter and maintain information in the patient's file. In the past, physicians would spend many hours entering and maintaining a patient's file. All information entered into a patient's file had to be handwritten into the file. The software allows the physician to easily enter data into the patient's file by using menu lists, pull down menus, anatomy photos, etc. which the physician can easily “click” to enter the data into the patient's file.

The software has the unique ability to build a chart note/medical record based on the diagnosis with which the patient presents. The physician selects the diagnosis. This builds the remainder of the chart note (the subjective and objective portions of the note). These portions are predetermined macros/strings in the form of the definition and description of the diagnosis. Each is electronically imported into the chart note from data tables. These components (the macros and strings) define and support the diagnosis and provide the supportive documentation for the selected diagnosis. This unique feature of providing the supportive and defining documentation (subjective and objective portions of the chart note/medical record) and verbage for a given diagnosis based on a single selection of the diagnosis greatly speeds the physician creating the medical record and makes the physician more accurate in his supportive documentation. Additionally the physician can select and bring into the chart note/medical record, any particular variable (facts and findings for a case) which he feels is appropriate and more specific/defining to the diagnosis. All of this functionality aids in specificity and uniqueness to the system.

Once the physician chooses the diagnosis from a constrained list of possibilities, the software allows the physician to select the treatment administered from another list of constrained choices. The list of treatments is generated from another database table that relates possible treatments to any giver diagnosis. The physician then selects the treatment he/she recommends for the next visit. The software will further allow the physician to document any specifics related to the diagnosis or treatments by selecting from another list of constrained choices. The list of findings is generated from another database table that relates supporting facts to diagnosis and treatment. The physician may further add any other observations which he/she feels may be pertinent. The notes may be added in a plurality of different manners. For example, the PA may allow for by voice dictation, manually writing/entering information into the PA, etc. Once the physician enters any notes, the physician may then select when the patient should be scheduled for the next visit. The physician may open up a new scheduling page on the PA to schedule the appointment.

The physician enters all of the above information into the PA. This information which is now stored on the PA may be downloaded/transmitted to the main database which is storing the information. The new record does not become committed, however, until either the physician reviews the chart note generated from the data or a specified time delay expires. The time delay is intended to give the physician time to review the record for accuracy. Once the record becomes committed it can not be changed in accordance with HIPPA requirements. Any corrections required beyond this time can only be made through addendum records.

If a consultation letter is required, the software will allow for one to be generated. A standard form letter template may be loaded by selecting that option on the PA 14 once an examination has been completed. Information may be added/entered into the letter from the PA. The letter may then be printed out, emailed, faxed etc. to the appropriate personnel. The consultation letter may be used as a work release letter, school release letter, etc.

If the patient is to be scheduled for surgery, a video clip can be played back for the patient's understanding and consent for treatment. The video clip is automatically selected based on the planned surgery choice. The video will aid the patient in understanding the proposed surgical procedure, thus allowing for a more thorough informed consent. The software may then list a set of instructions for the front office personnel. Instructions range from obtaining a patient consent form for the surgery, to handing a patient post treatment instruction sheets, to collecting co-payment for service. The set of instructions for patient “checkout” is automatically generated from the information recorded into the database and a set of rules specific to a given practice and specialty.

The software may further have a billing module that extracts all information related to patient care that can be billed, and generates a claim that can be sent to either the patient or the patient's insurance company. In accordance with one embodiment of the present invention, the software will use similar codes to that found on a medical insurance form. Thus, any information for work performed which is entered into the PA, can be directly inputted to a standard insurance form for processing by an insurance company. Thus, the software solves several of the problems discussed above. First, the software allows for one to easily prepare any patient's bill. Thus, much of the time and effort required by prior art methods is avoided. Second, the software for billing is compatible with the software for the starting and maintaining of medical records. Thus, one can easily transport information from the module for the starting and maintaining of medical records to the billing module.

The software may further have an inventory/office supply module. The inventory/office supply module maintains track of various supplies which are dispensed to a patient. Some of the supplies that may be dispensed on any given office encounter include, but is not limited to, splints, casts, fracture orthoses, pads bandages and dressings, orthotic devices, braces, etc. When a physician dispenses an office supply, the physician can enter this information in the PA. This information is stored in the database for tracking all office supplies. Thus, the software allows a physician to keep track of all office supplies. The software may further be programmed to provided alarms when a certain supply gets below a predetermined level. Thus, the software will give an indication that new supplies need to be ordered. The software may even be programmed to order the new supplies once the level gets below the predetermined level. Since all of the modules in the software are compatible, once some office supplies are recorded as being given to a patient, the software will input this information to the billing module for billing purposes.

The software may further have a prescription module. The prescription module allows the physician to select a desired medication for the patient based on the particular diagnosis. The software provides a listing of certain medications based on the inputted diagnosis generated from a database table relating treatment medicine to diagnosis. Once a desired medication is selected, the PA may either print this information so that a prescription form may be given to the patient or electronically transmit the prescription to the patient's preferred pharmacy where the prescription is to be filled. Thus, the prescription module will help to solve many of the problems associated with the prior art prescription systems.

The software may further have an outcome study module. The outcome study module gathers and analyzes all data stored in the software. The treatment data is stored in a separate and partitioned database such that there is no traceability to a particular patient. Thus, there are no patient confidentiality issues. Since multiple users may use the software, the software can perform outcome studies based on this information, allowing for regional, state, national or global outcome studies. The software employs a single database to gather treatment from any practice using the software to avail a very large data reservoir for outcome studies. Alternatively an individual practice can base an outcome study on its own separate data. Thus, the software will allow one to study effectiveness of various treatments and procedures.

Additional software functionality/capability is the electronic computer reading of radiographic images. The software program can evaluate a radiographic image that is scanned into the system. The system then automatically evaluates the image for various pathologies as well as analyzing critical radiographic angles on the image. These radiographic angles and diagnoses of pathology can then be used by the physician to determine best treatments of pathology, best surgical intervention and can be utilized to determine statistical analysis of the various pathologies and radiographic angles. All of this functionality can be used for but is not limited to aiding physicians in non biased, computerized system evaluations/analysis for diagnosis, treatment, creation of accurate supportive documentation for the chart note/medical record and statistical analysis (for outcome studies) of radiographic images.

While the invention has been particularly shown and described with reference to preferred embodiments thereof, it will be understood by those skilled in the art that the foregoing and other changes in form and details may be made therein without departing from the spirit and scope of the invention.

What is claimed is:

1. A method in a computer system for organizing a medical facility, the method comprising:
scheduling a visit by a patient;
entering and storing medical history of the patient in a database;
retrieving medical history of the patient from the database during the visit by the patient;
generating a patient chart note by entering a limited set of information;
diagnosing patient based on information entered; and
billing the patient.

2. The method of claim 1 further comprising maintaining an inventory of office supplies.

3. The method of claim 1 further comprising preparing a prescription form to be given to the patient.

4. The method of claim 3 further comprising transmitting prescription form to a desired pharmacy.

5. The method of claim 1 further comprising retrieving and organizing data entered into the computer system for performing outcome studies.

6. The method of claim 5 further comprising downloading data from different computer systems into the computer system for performing outcome studies.

7. The method of claim 1 wherein scheduling a visit by a patient further comprising inputting information into the computer system by the patient from a location remote from the computer system.

8. The method of claim 1 wherein entering and storing medical history of the patient in a database further comprises inputting information into the computer system by the patient from a location remote from the computer system.

9. The method of claim 1 wherein retrieving medical history of the patient from the database during the visit by the patient further comprises downloading the medical history of the patient from the computer system to a portable electronic device.

10. The method of claim 1 wherein diagnosing patient based on information entered into the computer system further comprises:
selecting a location from an anatomy photo;
choosing a diagnosis from a list constrained by selected location; and
selecting a treatment plan from a list constrained by selected diagnosis.

11. A computer readable medium containing instructions for controlling a computer system for organizing a medical facility by:
scheduling a visit by a patient;
entering and storing medical history of the patient in a database;
retrieving medical history of the patient from the database during the visit by the patient by downloading the medical history of the patient from the computer system to a portable electronic device;
diagnosing patient based on information entered into the computer system;
billing the patient;
maintaining an inventory of office supplies; and
preparing a prescription form to be given to the patient.

12. The method of claim 11 further comprising transmitting prescription form to a desired pharmacy.

13. The method of claim 11 further comprising retrieving and organizing data entered into the computer system for performing outcome studies.

14. The method of claim 13 further comprising downloading data from different computer systems into the computer system for performing outcome studies.

15. The method of claim 11 wherein scheduling a visit by a patient further comprising inputting information into the computer system by the patient from a location remote from the computer system.

16. The method of claim 11 wherein entering and storing medical history of the patient in a database further comprises inputting information into the computer system by the patient from a location remote from the computer system.

17. The method of claim 11 wherein diagnosing patient based on information entered into the computer system further comprises:
selecting a location from an anatomy photo on the portable electronic device;
choosing a diagnosis from a list displayed on the portable electronic device; and
selecting a treatment plan from a list displayed on the portable electronic device.

18. The method of claim 11 wherein billing the patient comprises:
transferring diagnosis data to a billing form; and
sending the billing form out for payment.

19. The method of claim 18 further comprising sending the billing form to a health insurance company of the patient for payment.

20. The method of claim 18 further comprising sending the billing form to the patient for payment.

21. The method of claim 1 wherein diagnosing patient further comprises forming a chart note/medical record based on the diagnosis.

22. The method of claim 21 wherein the forming of the chart note/medical record further comprises:
forming predetermined macros in the computer system which are definitions and descriptions of the diagnosis; and
importing the definitions and descriptions based on the diagnosis into the medical history of the patient.

23. The method of claim 1 wherein diagnosing patient further comprises:
scanning a radiographic image of the patient into the computer system;
evaluating the radiographic image.

24. The method of claim 23 further comprising measuring bone angle using image recognition software.
